(19) United States
(12) Patent Application Publication
(10) Pub. No.: US 2024/0369543 A1
(43) Pub. Date: Nov. 7, 2024

(54) MEASURING REAGENT FOR CROSS-LINKED N-TELOPEPTIDE OF TYPE I COLLAGEN, PREPARATION METHOD THEREOF, AND IMMUNOASSAY METHOD USING SAME

(71) Applicant: SEKISUI MEDICAL CO., LTD., Tokyo (JP)

(72) Inventors: Tomo SHIMIZU, Tokyo (JP); Tomohide ASAI, Tokyo (JP)

(73) Assignee: SEKISUI MEDICAL CO., LTD., Tokyo (JP)

(21) Appl. No.: 18/688,434

(22) PCT Filed: Oct. 18, 2022
(86) PCT No.: PCT/JP2022/038705

§ 371 Date: Mar. 1, 2024

(30) Foreign Application Priority Data
Oct. 20, 2021 (JP) 2021-171929

(51) Int. Cl. G01N 33/53; C07K 16/18; G01N 33/68

(52) U.S. Cl. CPC G01N 33/53; C07K 16/18; G01N 33/6854

(57) ABSTRACT

A measurement reagent for a cross-linked N-telopeptide of type I collagen containing uric acid or its salt. This reagent is easier to handle and enables NTx measurement with higher accuracy.

### TECHNICAL FIELD

The present invention relates to a measurement reagent for cross-linked N-telopeptide of type I collagen and an immunoassay method using the same. The present invention also relates to a method for preparing a measurement reagent for cross-linked N-telopeptide of type I collagen.

### BACKGROUND ART

Cross-linked N-telopeptide of type I collagen (hereinafter also referred to as NTx) is a bone-derived degradation product of type I collagen. NTx is produced when type I collagen is digested by Cat K during the process of bone resorption. After being produced, NTx is excreted into the blood and/or urine.

The level of NTx increases as the bone resorption advances. Therefore, NTx serves as an index that directly reflects bone resorption. This allows NTx to be used as a marker for diagnosing osteoporosis or determining therapeutic efficacy.

Patent Literature 1 describes that the rate of bone resorption is measured by measuring NTx in urine. Patent Literature 2 describes monoclonal antibody 1H11 that binds to NTx. Further, Patent Literature 2 also describes an epitope recognized by the monoclonal antibody 1H11.

A kit for measuring NTx by ELISA method using a monoclonal antibody is also commercially available (Non-Patent Literature 1). However, it has been desired to develop a reagent for measuring NTx, which is easier to handle and enables measurement with higher accuracy.

### CITATION LIST

### Patent Literature

Patent Literature 1: Japanese Unexamined Patent Application Publication (Translation of PCT Application) No. H3-500818

Patent Literature 2: Japanese Unexamined Patent Application Publication (Translation of PCT Application) No. H11-505804

### Non Patent Literature

Non-Patent Literature 1: Package insert of in vitro diagnostic drug “Osteomark (registered trademark) Kit for Cross-linked N-telopeptides of Type I Collagen” (Abbott Diagnostics Medical Co., Ltd.)

Non-Patent Document 2: J. Bone Miner. Res 1992, 7 (11), 1251-1258 A specific immunoassay for monitoring human bone resorption: quantitation of type I collagen cross-linked N-telopeptides in urine

### SUMMARY OF INVENTION

### Technical Problem

An object of the present invention is to provide a reagent for measuring NTx, which is easier to handle and enables NTx measurement with higher accuracy.

### Solution to Problem

In the measurement kit described in Non-Patent Literature 1, when a urine sample contains NTx at a high concentration exceeding the measurement upper limit, the urine sample is diluted with urine having a known NTx concentration and then measurement is carried out. In this case, it is necessary to divide the measurement results by the NTx content in the urine used for dilution or subtract the NTx content from the measurement results. When the present inventors diluted a urine sample with urine of known NTx concentration to measure the concentration of NTx in the sample, it was revealed that the amount of uric acid present in the measurement system during the NTx measurement affected the measured value of NTx (Comparative Example 1). Since the concentration of uric acid in urine for dilution varies, there are cases where the measured value of NTx is affected by the amount of uric acid contained in the urine samples or urine for dilution, thereby hindering accurate measurement of NTx.

The present inventors have made extensive studies and found that by using a measurement reagent for cross-linked N-telopeptide of type I collagen containing uric acid or its salt, it becomes possible to perform an immunoassay with easy operation and with high accuracy in NTx measurement. Based on this finding, the present invention has been completed.

Specifically, the present invention is as follows.
<1> A measurement reagent for a cross-linked N-telopeptide of type I collagen containing uric acid or its salt.
<2> The measurement reagent according to <1>, which is a reagent for immunoassay and is in a liquid form.
<3> The measurement reagent according to <1> or <2>, which is a measurement reagent for measuring a cross-linked N-telopeptide of type I collagen in urine.
<4> The measurement reagent according to any one of <1> to <3>, which is a specimen diluent, an antibody diluent, or a particle suspension.
<5> The measurement reagent according to any one of <1> to <4>, wherein a concentration of uric acid or its salt is 0.001 to 10% by mass, based on the measurement reagent.
<6> The measurement reagent according to any one of <1> to <5>, which further comprises a buffer.
<7> An immunoassay method for a cross-linked N-telopeptide of type I collagen, comprising:
   a contacting step of bringing a biological sample into contact with uric acid or its salt, or a reagent containing uric acid or its salt.
<8 The immunoassay method according to <7>, wherein the contacting step is a step of bringing the biological sample into contact with the reagent containing uric acid or its salt.
<9> The immunoassay method according to <7> or <8>, wherein the biological sample is urine.
<10> The immunoassay method according to any one of <7> to <9>, which further comprises:
   a step of bringing the biological sample into contact with an antibody that binds to a cross-linked N-telopeptide of type I collagen or an antibody fragment thereof; and
   a step of measuring a signal derived from a labeling substance indirectly or directly bound to the antibody or the antibody fragment thereof.
<11> The immunoassay method according to any one of <7> to <10>, wherein a concentration of uric acid during antibody-antigen reaction is 0.0001 to 1% by mass.
<12> The immunoassay method according to any one of <7> to <11>, which does not comprise a step of dividing the signal or a measured value of the cross-linked N-telopeptide of type I collagen based on the signal by an amount of another cross-linked N-telopeptide of type I collagen derived from a source other than the biological sample, or subtracting the amount of the another cross-linked N-telopeptide of type I collagen from the signal or the measured value of the cross-linked N-telopeptide of type I collagen.
<13> A method for preparing a measurement reagent for a cross-linked N-telopeptide of type I collagen, comprising:
   adding uric acid or its salt to a solvent.
<14> The method according to <13>, wherein the solvent is a buffer.

### Advantageous Effects of Invention

The present invention can provide a reagent for measuring NTx, which is easier to handle and enables NTx measurement with higher accuracy.

### BRIEF DESCRIPTION OF DRAWINGS

FIG. 1 is a diagram showing the chemical structure of NTx.

FIG. 2 is a graph showing the correlation between ECLIA measurements and Osteomark measurements when dilution measurements were performed using a biological sample diluted with urine.

FIG. 3 is a graph showing the correlation between ECLIA measurements and Osteomark measurements when dilution measurements were performed using a biological sample diluted with uric acid.

FIG. 4 is a graph showing the results of comparison of measurements between dilution with urine and dilution with a uric acid diluent in Osteomark measurements.

### DESCRIPTION OF EMBODIMENTS

Hereinbelow, the descriptions are given separately for various aspects of the invention, but the descriptions, definitions of terms, and embodiments described for a particular aspect are also applicable to the other aspects.

### 1. Measurement Reagent for Cross-Linked N-Telopeptide of Type I Collagen

### (Cross-Linked N-Telopeptide of Type I Collagen (NTx))

NTx is a bone-derived degradation product of type I collagen. NTx is produced when type I collagen is digested by Cat K during the process of bone resorption. After being produced, NTx is excreted into the blood and/or urine.

The level of NTx increases as the bone resorption advances. Therefore, NTx serves as an index that directly reflects bone resorption. This allows NTx to be used as a marker for diagnosing osteoporosis or determining therapeutic efficacy.

In addition to diagnosing osteoporosis or determining therapeutic efficacy, NTx is also measured for the following purposes.
Determination of the suitability for surgery for primary hyperparathyroidism
Evaluation of therapeutic effects after hyperparathyroidism surgery
Indicators of bone metastasis of malignant tumors and indicators of the degree of progression of bone metastatic lesions

In this context, the measurement reagent of the present invention is not limited to those used for measurement for the above purposes.

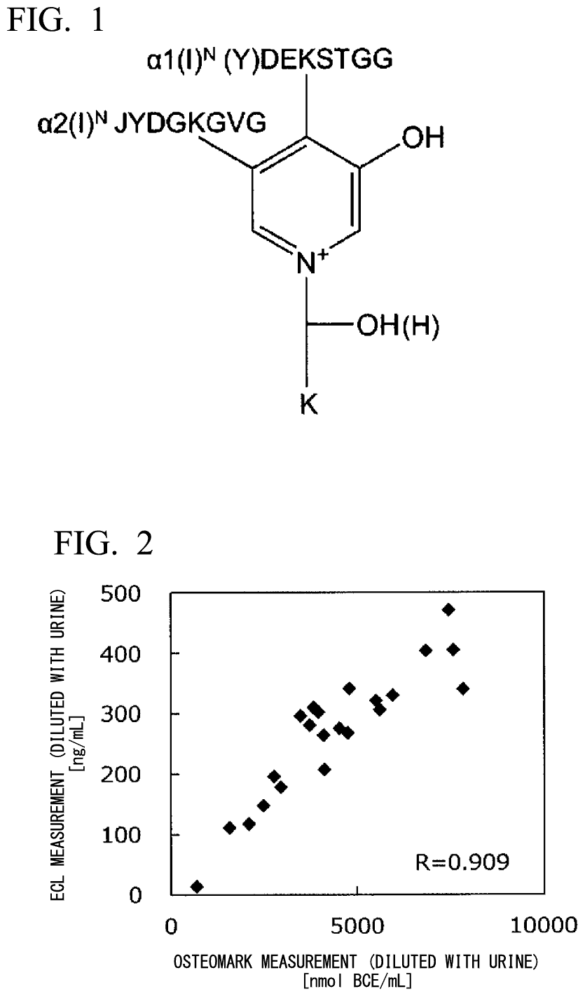

NTx has a structure shown in FIG. 1. In NTx, an α1 chain and an α2 chain are bonded to a pyridinium crosslinked structure.

### (Uric Acid or its Salt)

The measurement reagent of the present invention comprises uric acid. Uric acid is an organic compound represented by a molecular formula C₅H₄N₄O₃ (CAS RN 69-93-2). In the present invention, salts of uric acid such as sodium hydrogen urate, potassium hydrogen urate, disodium urate, dipotassium urate, and calcium urate can be used as long as uric acid is produced by ionization in a solution and the effects of the present invention can be obtained.

In the measurement reagent of the present invention, the concentration of uric acid or its salt can be appropriately adjusted to a desired concentration during antigen-antibody reaction, but is, for example, 0.001 to 10% by mass, preferably 0.01 to 5% by weight, more preferably 0.02 to 2% by weight, even more preferably 0.05 to 1% by weight, based on the measurement reagent. When a salt of uric acid is used, the above concentration means the concentration of the uric acid moiety excluding potassium, sodium, and the like.

In the present specification, the amount of uric acid can be measured by an “enzymatic method” using uricase, which is a urate oxidase.

### (Biological Sample)

Examples of the “biological sample” in the present specification, which contains cross-linked N-telopeptide of type I collagen, include solid tissues and body fluids derived from living bodies.

The biological sample is preferably a body fluid, examples which include blood, serum, plasma, urine, tears, ear discharge, prostatic fluid, and respiratory secretions. The biological sample is more preferably urine or serum, even more preferably urine.

Examples of subjects from which the biological sample is to be collected include humans or animals (e.g., monkeys, dogs, and cats), of which humans are preferable. The biological sample may be a biological sample as it is taken from a subject, or may be a sample obtained by subjecting a collected biological sample to treatments such as dilution and concentration that are usually performed. The person who collects and prepares a biological sample may or may not be identical to the person who performs the immunoassay. Further, the biological sample may be one collected or prepared during implementation of the immunoassay, or one previously collected or prepared and stored. The biological sample may be one collected from a subject suffering from a metabolic disease that causes increased bone resorption, such as osteoporosis, primary hyperparathyroidism, or a malignant tumor with suspected bone metastases (particularly breast, lung, or prostate cancer).

### (Buffer)

In the present specification, the term “buffer” means a solution having a pH-buffering effect.

Non-limiting examples of buffers that can be used as appropriate in the present invention include known buffers such as PBS (phosphate buffered saline), MES (2-(N-morpholino)ethanesulfonic acid), PIPES (piperazine-N,N′-bis(2-ethanesulfonic acid), ACES (N-(2-acetamido)-2-aminoethanesulfonic acid), ADA (N-(2-acetamido)iminodiacetic acid), Bis-Tris(2,2-bis(hydroxyethyl)-(iminotris-(hydroxymethyl)-methane), Tris (tris(hydroxymethyl)aminomethane), MOPS (3-morpholinopropanesulfonic acid), HEPES (4-(2-hydroxyethyl)-1-piperazineethanesulfonic acid), a citric acid buffer, a glycine buffers, a borate buffer, and a phosphate buffer.

The buffer used in the present invention is preferably HEPES.

The concentration of the buffer is not particularly limited as long as the effects of the present invention can be obtained, and is, for example, 1 to 500 mM, 5 to 400 mM, 10 to 300 mM, or 15 to 100 mM.

In the measurement reagent of the present invention, the amount of the buffer may be, for example, 90% by mass or more, 92% by mass or more, 95% by mass or more, 97% by mass or more, or 99% by mass or more, with respect to the measurement reagent.

### (Measurement Reagent)

In the present specification, the term “measurement reagent” means a reagent used for measuring NTx. The measurement reagent encompasses a specimen diluent, a particle suspension, an antibody diluent, a calibrator, a calibration sample, a control solution, and the like. The measurement reagent is preferably a sample diluent or a particle suspension. The term “sample diluent” means a reagent added to a biological sample to dilute the biological sample. The term “antibody diluent” means a reagent for diluting antibodies. The sample diluent and antibody diluent may be used only for either the sample or the antibody, or for both. The term “particle suspension” means a reagent for suspending and preserving particles such as magnetic particles or latex particles when these particles are used for measurement.

The pH of the measurement reagent of the present invention is, for example, 4.0 to 11.0, 5.0 to 10.0, 6.0 to 9.5, or 6.4 to 8.6. Adjustment of pH can be carried out using pH adjusting reagents well known to those skilled in the art, such as sodium hydroxide or hydrochloric acid.

The measurement reagent of the present invention does not include urine itself or diluted urine for diluting a biological sample. Those in which the uric acid content is adjusted by adding uric acid or a salt thereof to urine are within the scope of the present invention.

The NTx content in the measurement reagent of the present invention is preferably 100 ng/mL or less, more preferably 50 ng/mL or less, even more preferably 10 ng/ml or less, even more preferably 5 ng/ml or less, even more preferably 1 ng/ml or less, even more preferably 0.1 ng/ml or less, and the measurement reagent is most preferably NTx-free.

The measurement reagent of the present invention may be in any form, but is preferably in a liquid form. The measurement reagent of the present invention is preferably a reagent for immunoassay.

It is preferable that uric acid or its salt is included in the measurement reagent at the time of sale. However, uric acid or its salt may be contained in a separate container from that containing the measurement reagent, and the person performing the measurement may add uric acid or its salt to the measurement reagent.

The measurement reagent of the present invention can be prepared by adding uric acid or its salt to a solvent such as a buffer.

### (Storage Container)

The measurement reagent of the present invention is preferably packed in a storage container. The material of the storage container is not particularly limited as long as the effects of the present invention can be achieved and the container can be sealed tightly.

### Immunoassay Method for Cross-Linked N-Telopeptide of Type I Collagen

The “immunoassay method” is a method of measuring the level of a substance contained in a biological sample using the reaction between an antigen and an antibody. The term “level” encompasses the amount, concentration, or determination of presence or absence of a substance.

Examples of the immunoassay method include, but not limited to, electrochemiluminescence immunoassay (ECLIA method), enzyme-linked immunosorbent assay (ELISA), latex immunoturbidimetric assay (LTIA method), chemiluminescence immunoassay, immunochromatography, and immunofluorescence. In the present invention, the immunoassay method is preferably ELISA or ECLIA.

In the present invention, the immunoassay method can be an in vivo or in vitro immunoassay method. Further, a sensitizer may also be used to enhance sensitivity.

In the immunoassay method of the present invention, when a monoclonal antibody is used as the antibody, it is preferable to use only a single type of monoclonal antibody for immunoassay of NTx, for example, by employing the competition method described below. When using two types of monoclonal antibodies, the two types of monoclonal antibodies preferably recognize different epitopes. To recognize different epitopes in this context means that the amino acid sequences recognized as epitopes by the two monoclonal antibodies do not overlap. When there are multiple epitopes, one of the multiple epitopes only needs to be different from one of the multiple epitopes of the other antibody. For example, a sandwich system can be constructed using the following two types of antibodies: an antibody that binds to a peptide fragment with an amino acid sequence represented by JYDGKGVG (SEQ ID NO: 1) and an antibody that binds to other structures of NTx.

### (Antibody)

In the immunoassay method of the present invention, monoclonal antibodies or polyclonal antibodies can be used as antibodies. Preferably, monoclonal antibodies are used. Antibody fragments possessing the functions of antibodies can also be used. As the antibody, any antibody that binds to NTx can be used without limitation, but it is preferable to use an antibody that binds to a peptide fragment having an amino acid sequence represented by JYDGKGVG (SEQ ID NO: 1), more preferably an antibody that binds to a peptide fragment having an amino acid sequence represented by QYDGK(C)GVG (SEQ ID NO: 2) as well as the peptide fragment having an amino acid sequence represented by JYDGKGVG (SEQ ID NO: 1). The (C) in “QYDGK(C)GVG” is bonded to the side chain of K. That is, the “(C)” and the first “G” in “GVG” are both bonded to K.

### (Labeling Substance)

It is preferable that a labeling substance is bound to the antibody. By measuring the intensity of the signal generated by the labeling substance, the amount of NTx in the biological sample can be measured. The labeling substance may be directly bound to the antibody or indirectly bound to the antibody via a secondary antibody. Hereinbelow, an antibody to which a labeling substance is bound may be referred to as a labeled antibody. Examples of labeling substances for preparing the labeled antibody include metal complexes, enzymes, insoluble particles, fluorescent substances, chemiluminescent substances, electrochemiluminescent substances (such as ruthenium complexes), biotin, avidin, radioactive isotopes, colloidal gold particles, and colored latex.

In the immunoassay method of the present invention, as the labeling substance, it is preferable to use an electrochemiluminescent substance, and it is more preferable to use a ruthenium complex. When an enzyme such as horseradish peroxidase (HRP) or alkaline phosphatase (ALP) is used as a labeling substance, the concentration of NTx in a biological sample can be measured using a specific substrate for the enzyme. When the enzyme is HRP, for example, O-phenylenediamine (OPD) or 3,3′,5,5′-tetramethylbenzidine (TMB) can be used as a substrate, and in the case of ALP, p-nitrophenyl phosphate and the like can be used as a substrate.

In the context of the present specification, the process of physically or chemically supporting an antigen or antibody on a solid phase or the state of an antigen or antibody being supported on a solid phase is sometimes referred to as “immobilization” or “solid-phase immobilization”. The term “assay”, “detection” or “measurement” also encompasses proving the presence of NTx and quantification of NTx.

When immobilizing an antigen on a solid phase, the antigen is not particularly limited as long as it binds to the antibody. The antigen may be, for example, NTx, the alpha chain of NTx, or a peptide fragment having an amino acid sequence represented by JYDGKGVG (SEQ ID NO: 1).

The solid phase to be used may be a solid phase composed of a polymer substrate such as polystyrene resin, an inorganic substrate such as glass, a polysaccharide substrate such as cellulose or agarose, or the like. The shape of the solid phase is not particularly limited, and any appropriate shape may be chosen, for example, from a plate shape (e.g., microplate or membrane), a bead or particulate shape (e.g., latex particles, magnetic particles), or a cylindrical shape (e.g., test tube).

The present specification includes descriptions indicating that an antibody or an antibody fragment thereof “reacts with”, “recognizes” or “binds to” a specific substance or amino acid sequence, which however are used interchangeably. Whether or not an antibody “reacts with” an antigen (compound) can be determined through antigen-immobilized ELISA, competitive ELISA, sandwich ELISA, or the like. Alternatively, a method using the principle of surface plasmon resonance (SPR method) can also be used. The SPR method can be performed using equipment, sensors and reagents commercially available under the name Biacore (registered trademark).

For example, when performing the same operation as the screening method of Preparation Example 1 (antigen-immobilized ELISA) described below, a peptide fragment with its absorbance significantly increased compared to a negative control without addition of a peptide fragment can be evaluated as being bound to the antibody.

The concentration of the antibody of the present invention in the measurement system can be adjusted as appropriate depending on the immunoassay method or the type of biological sample. For example, the concentration may be 0.1 ng/ml to 100 μg/mL.

### (Step of Bringing a Biological Sample Into Contact With Uric Acid or its Salt, or a Reagent Containing These)

In the present invention, the term “contact” means to make physical contact, and the specific means thereof is not limited. For example, uric acid or its salt may be directly added to a measurement system containing a biological sample, or a reagent containing uric acid or its salt may be added to a measurement system. In this context, the order of adding the biological sample, uric acid or its salt, or a reagent containing these, and an antibody that binds to NTx or an antibody fragment thereof to the measurement system is limited as long as the effects of the present invention can be obtained. The effects of the present invention can be obtained by performing an antibody-antigen reaction between NTx contained in a biological sample and an antibody that binds to NTx or an antibody fragment thereof in the presence of uric acid.

The step of contacting the biological sample with uric acid, its salt, or a reagent containing these does not include a step of contacting the biological sample with urine or a diluted solution thereof.

The concentration of uric acid during the antibody-antigen reaction is not limited as long as the effects of the present invention can be obtained, but the concentration is, for example, 0.0001 to 1% by mass, preferably 0.0005 to 0.5% by mass, more preferably 0.001 to 0.2% by mass, more preferably 0.002 to 0.1% by mass.

The immunoassay method of the present invention preferably does not include a step of dividing the signal or the NTx measurement based on the signal by the amount of NTx originating from a source other than the biological sample, or subtracting the amount of NTx originating from the other source from the signal or the NTx measurement based on the signal. The step of division or subtraction that is preferably not included in the immunoassay method of the present invention is a step of dividing or subtracting with respect to the obtained signal, and a step of dividing or subtracting with respect to the obtained measurement value.

In the conventional method for measuring NTx in which a specimen is diluted with urine, it is necessary to divide the measurement values by the NTx content of the urine used for dilution or subtract the NTx content of the urine used for dilution from the measurement values. In the immunoassay method of the present invention, it is possible to accurately measure the amount of NTx without the step of dividing the measurement values or subtracting from the measurement values. However, the present invention does not exclude such a step of division or subtraction from the immunoassay method of the present invention.

Hereinbelow, the procedure and principle of measurement are explained, taking as examples competitive ELISA and competitive ECLIA as immunoassay methods. The following descriptions are presented for illustrative purpose only with respect to the measurement procedure and principle for one embodiment of the present invention, and by no means limit the scope of the present invention. In each of the immunoassay methods described below, methods known in the art including those described above can be used without any limitation with respect to specific methods such as the method for immobilizing the antibody on the solid phase, the method for binding the antibody to the labeling substance, and the type of labeling substance.

The immunoassay method of the present invention may be, for example, competitive ELISA, which is a competitive method, including steps (1) to (3) as described below. The order of performing the steps (1) to (3) is not limited.
(1) A biological sample to be analyzed is added to a microplate on which NTx or its peptide fragment for solid-phase binding is immobilized. The biological sample is diluted in advance with a sample diluent containing uric acid.
(2) An antibody that binds to an enzyme-labeled NTx or its peptide fragment is added to the microplate.
(3) A substrate for the enzyme is added, and a signal attributable to the enzymatic reaction is measured.

The signal intensity decreases when NTx is present in the biological sample, and competition occurs between the following two reactions: a reaction between NTx in the biological sample and the antibody that binds to NTx or its peptide fragment, and a reaction between the NTx or its peptide fragment immobilized on the solid-phase, and the antibody that binds to NTx or its peptide fragment. Further, the antibody may be labeled with biotin instead of an enzyme. In this case, streptavidin labeled with an enzyme may be bound to the biotin. Then, a chromogenic signal generated by addition of OPD as a substrate can be measured.

A secondary antibody can also be used in competitive ELISA. In the present specification, the “secondary antibody” is an antibody that specifically recognizes an antibody that binds to NTx. When using a secondary antibody, the following procedures (1) to (5) can be adopted.
(1) A biological sample to be analyzed is added to a microplate on which NTx or its peptide fragment for solid-phase binding is immobilized. The biological sample is diluted in advance with a sample diluent containing uric acid.
(2) An antibody that binds to NTx or its peptide fragment is added to the microplate.
(3) Further, an enzyme-labeled secondary antibody is added.
(4) A substrate is added for color development.
(5) The signal attributable to the substrate is measured using a plate reader or the like.

Electrochemiluminescence immunoassay (ECLIA) means a method in which a labeling substance is caused to emit light by application of electric current, and the amount of light emitted is detected to measure the amount of a target substance to be detected. A ruthenium complex can be used as a labeling substance in the electrochemiluminescence immunoassay. An electrode is placed on a solid phase (such as a microplate), and radicals are generated on the electrode to excite the ruthenium complex to emit light. Then, the amount of light emitted from this ruthenium complex can be detected.

The immunoassay method of the present invention may be, for example, competitive ECLIA, which is a competitive method, including steps (1) to (3) as described below. The order of performing the steps (1) to (3) is not limited.
(1) A biological sample to be analyzed is added to a measurement system containing magnetic particles on which NTx or its peptide fragment for solid-phase binding is immobilized. The biological sample is diluted in advance with a sample diluent containing uric acid.
(2) An antibody that binds to NTx or its peptide fragment and is labeled with an electrochemiluminescent substance, preferably a ruthenium complex, is added to the measurement system.
(3) The intensity of luminescence derived from the luminescent label is measured.

The luminescence intensity decreases when NTx is present in the biological sample, and competition occurs between the following two reactions: a reaction between NTx in the biological sample and the antibody that binds to NTx or its peptide fragment, and a reaction between the NTx or its peptide fragment for solid-phase binding, which is immobilized on the magnetic particles, and the antibody that binds to NTx or its peptide fragment.

In the immunoassay method of the present invention, it is preferable to use two types of monoclonal antibodies that recognize different epitopes. For convenience, one of the monoclonal antibodies may be referred to as “first monoclonal antibody”, while the other one of the monoclonal antibodies may be referred to as “second monoclonal antibody”.

When the first monoclonal antibody is a labeled antibody and the second monoclonal antibody is a solid-phase antibody, the immunoassay method of the present invention may include the following steps (1) to (3).
(1) A step of contacting a biological sample with a first monoclonal antibody having a labeling substance bound thereto to form a first complex (containing Ntx in the biological sample or a peptide fragment thereof, the labeling substance, and the first monoclonal antibody).
(2) A step of contacting the first complex with a second monoclonal antibody to form a second complex (containing Ntx in the biological sample or a peptide fragment thereof, the labeling substance, the first monoclonal antibody, and the second monoclonal antibody).
(3) A step of measuring a signal attributable to the labeling substance.

The second complex contains a labeling substance. For signal measurement, a measurement method well known in the art can be employed depending on the labeling substance. The signal measurement may be performed using a measuring instrument, or may be performed visually.

The immunoassay method of the present invention may further include the following steps, if necessary:
step of pretreating a biological sample,
step of immobilizing NTx or its peptide fragment for solid-phase binding on a solid phase,
B/F washing step of washing and removing antibodies that are not bound to the NTx or its peptide fragment for solid-phase binding as well as the biological sample,
step of calculating the NTx concentration in the biological sample from the measured luminescence intensity, based on the luminescence intensity in a measurement of a sample containing NTx in a known concentration, and/or
comparison step of comparing the calculated NTx concentration in the biological sample with a first threshold value.

Examples of pretreatments include filtration of the biological sample and dilution of the biological sample with a sample diluent.

The first threshold may be appropriately set in consideration of sensitivity, type of the biological sample, and purpose of the NTx measurement. When the biological sample is urine, the following values can be adopted as the first threshold value depending on the purpose of measurement.
Indication for parathyroidectomy: 200 nM BCE/mM-Cre or higher
Indicator of bone metastasis of malignant tumors (breast cancer, lung cancer, prostate cancer): 100 nM BCE/mM-Cre or higher
Indicator of increased bone resorption: 55 nM BCE/mM-Cre or higher
Indicator of osteoporosis drug treatment (indicator of high risk of fracture): higher than 54.3 nM BCE/mM-Cre
Indicator of osteoporosis drug treatment (indicators of high risk of bone loss): 35.3 nM BCE/mM-Cre or higher

The first threshold may be a numerical range. The first threshold being a numerical range means that the specified range include a specific threshold, and the determination of whether the measured value is larger or smaller than the specific threshold allows determination of the presence or absence of a disease, etc.

When the first threshold is a numerical range, the first threshold may be in a range between 1.0 and 300 nM BCE/mM-Cre, between 5.0 and 250 nM BCE/mM-Cre, or between 7.0 and 220 nM BCE/mM-Cre.

In the immunoassay method of the present invention, when the signal intensity is higher than the first threshold, the method may include a step of determining that a subject is suffering from a metabolic disease that causes increased bone resorption, such as osteoporosis or primary hyperparathyroidism, or that bone metastasis is suspected in a subject suffering from a malignant tumor (particularly breast cancer, lung cancer, or prostate cancer).

In the immunoassay method of the present invention, when the signal intensity is lower than the first threshold, the method may include a step of determining that a subject is not suffering from a metabolic disease that causes increased bone resorption, such as osteoporosis or primary hyperparathyroidism, or that bone metastasis is not suspected in a subject suffering from a malignant tumor (particularly breast cancer, lung cancer, or prostate cancer).

Based on the NTx concentration in a biological sample calculated by the immunoassay method of the present invention, the therapeutic effect of a specific drug on a subject suffering from osteoporosis can be determined. In this instance, the immunoassay method of the present invention may further include the following step(s) in addition to the steps described above:
a step of administering a specific drug to the subject, and/or
a step of comparing the NTx concentration in the biological sample collected from the subject with a second threshold.

In this instance, the second threshold may be appropriately set in consideration of sensitivity of the immunoassay method, type of the biological sample, and purpose of the NTx measurement. The second threshold may be the measured NTx in the subject prior to administration of a specific drug to the subject.

The immunoassay method of the present invention may include a step of determining that a specific drug has a therapeutic efficacy when the signal intensity is lower than the second threshold, or a step of determining that a specific drug has no therapeutic efficacy when the signal intensity is higher than the second threshold.

In the determination of the therapeutic efficacy, the therapeutic efficacy may be monitored by conducting measurement every few days.

Examples of the specific drugs include bisphosphonate preparations, anti-RANKL antibodies (denosumab), and calcium preparations.

Hereinbelow, the present invention is described in detail with reference to examples, which however should not be construed as limiting the present invention. In the following description, the unit “%” refers to “% by mass”, unless otherwise specified.

### EXAMPLES

### Preparation Example 1 Preparation of Monoclonal Antibody

### Preparation of Immunogen and Antigen for Screening

Inject Maleimide-Activated Ovalbumin (manufactured by Thermo Scientific, CAT No. 77126) was dissolved in 0.2 mL of purified water to prepare 10 mg/ml of Maleimide-Activated Ovalbumin solution. 2 mg of peptide QYDGK (C) GVG ((C) is bonded to the side chain of K, Nx-2 peptide) was dissolved in 0.2 mL of PBS to obtain a 10 mg/ml peptide solution. The prepared Maleimide-Activated Ovalbumin solution and the peptide solution were mixed and stirred at room temperature for 2 hours. The resulting reaction solution was dialyzed against PBS to obtain a peptide (immunogen) to which Ovalbumin was bound via the thiol group of cysteine.

### Immunization Method

20 μL of the immunogen was mixed with Freund's Complete Adjuvant (manufactured by Difco Laboratories) and the resulting was used to immunize a 6-week-old F344/Jc1 rat subcutaneously on its back or footpad. Two weeks later, 20 μL of the immunogen was mixed with Freund's Incomplete Adjuvant (manufactured by Difco Laboratories) and the resulting was used to immunize the rat subcutaneously on its back or footpad. The same procedure was repeated continually every two weeks. In the third and subsequent immunizations, the individuals having shown a sufficient increase in antibody titer were intraperitoneally immunized with the immunogen diluted in PBS. One to three days after the intraperitoneal immunization, spleen cells, iliac lymph node cells, and inguinal lymph node cells were collected and fused with myeloma cells SP2/0 by electrofusion method. The fused cells were cultured in a 96-well plate, and the culture supernatant was collected 7 or 8 days after the fusion, followed by screening by antigen-immobilized ELISA described below. The strains that reacted to the Nx-2 peptide were selected and cloned.

### Screening Method (Antigen-Immobilized ELISA)

Inject Maleimide-Activated BSA (manufactured by Thermo Scientific, CAT No. 77126) was dissolved in 0.2 mL of purified water to prepare 10 mg/ml of Maleimide-Activated BSA solution. 2 mg of Nx-2 peptide was dissolved in 0.2 mL of PBS to obtain a 10 mg/ml peptide solution. The prepared Maleimide-Activated BSA solution and the peptide solution were mixed and stirred at room temperature for 2 hours. The resulting reaction solution was dialyzed against PBS to obtain a peptide to which BSA was bound via the thiol group of cysteine (i.e., Nx-2 peptide-bound BSA). 50 μL of Nx-2 peptide-bound BSA dissolved in PBS at a concentration of 0.1 μL/mL was dispensed into each well of a 96-well plate and allowed to stand at room temperature for 2 hours. After washing each well three times with PBST, 100 μL of blocking solution (1% BSA-PBST) was dispensed into each well and the resulting was allowed to stand at room temperature for 1 hour. After removing the blocking solution from each well, 50 μL of the culture supernatant was dispensed into each well and the resulting was allowed to stand at room temperature for 1 hour. After washing each well three times with PBST, 50 μL of HRP-labeled goat anti-rat IgG (Fc) polyclonal antibody (manufactured by Southern Biotech) diluted 10,000 times with a blocking solution was dispensed into each well and the resulting was allowed to stand at room temperature for 1 hour. After washing each well three times with PBST, 50 μL of an OPD coloring solution was dispensed into each well and the resulting was allowed to stand at room temperature for 10 minutes. 50 μL of a stop solution was added to each well to stop the reaction. Absorbance at a wavelength of 492 nm was measured using a plate reader to determine the reactivity between the Nx-2 peptide-bound BSA and the antibody. Three strains of antibodies that react with Nx-2 peptide and NTx were established. The S88230R antibody was selected from the established antibodies, and ascites was prepared using antibody-producing cells, which was then purified using a protein G column to be used in subsequent tests.

### Preparation Example 2 Preparation of Various Reagents for ECLIA Measurement Using S88230R Antibody

### Preparation of Biotin-Labeled Nx7 Peptide

2 mg of a peptide (Nx7 peptide) having an amino acid sequence JYDGKGVG was dissolved in 1 mL of 100 mM PBS with a pH of 7.5 to prepare an Nx7 peptide solution. 12.7 mg of Ez-Link NHS-PEG12-Biotin (manufactured by Thermo Scientific) was dissolved in 0.041 mL of dehydrated DMF, and the entire amount of the resulting was added to the Nx7 peptide solution. After stirring on ice for 3 hours, the resulting was purified by reverse phase chromatography to remove unreacted biotin reagent, thereby obtaining a biotin-labeled Nx7 peptide solution.

### Preparation of Nx7 Peptide-Immobilized Magnetic Particles

After washing 100 μL of 30 mg/mL Streptavidin/solid-phase magnetic particles three times with PBS, followed by completely removing PBS, 600 μL of a biotin-labeled Nx7 peptide solution dissolved in PBS at a concentration of 3.3 μg/mL was added, and the resulting was stirred at 25° C. for 2 to 3 hours. After washing the obtained magnetic particles three times with magnetic particle storage solution (50 mM HEPES, 1% BSA, 150 mM NaCl, 2 mM EDTA-4Na, 0.01% Tween 20, pH 7.2), the magnetic particles were suspended in 300 μL of magnetic particle storage solution to obtain a suspension of Nx7 peptide-immobilized magnetic particles. The Nx7 peptide-immobilized magnetic particle suspension was adjusted to a concentration of 0.05 mg/mL with R2 reagent (50 mM HEPES, 1% BSA, 150 mM NaCl, 2 mM EDTA-4Na, 0.01% Tween 20, pH 7.2), 5 and subjected to ECLIA measurement.

### Preparation of Ruthenium-labeled S88230R Antibody

To 1 mL of 1 mg/mL S88230R antibody, 68 μL of 10 mg/mL Ruthenium (II) Tris(bipyridyl)-NHS Ester (dissolved in DMSO) was added, followed by stirring at room temperature for 30 minutes. Then, 50 μL of 2 mol/L glycine was added, and the resulting was stirred at room temperature for 10 minutes. From the obtained reaction solution, unreacted antibodies and ruthenium complexes were removed using Sephadex G-25 to obtain a ruthenium-labeled S88230R antibody.

### Preparation of Calibrator and Sample

The Nx7 peptide was dissolved in R1 reagent (50 mM HEPES, 1% BSA, 150 mM NaCl, 2 mM EDTA-4Na, 0.01% Tween 20, non-specific inhibitor, pH 7.2) to a concentration of 250 ng/mL to obtain a standard product. Solutions in which the standard product was diluted 1, 2, 4, 8, 16, 32, 64, and 128 times with the R1 reagent were prepared and used as calibrators. As samples, either undiluted urine specimens or urine specimens diluted with predetermined solutions were used.

### Analysis Example 1 ECLIA Measurement Procedure Using S88230R Antibody

Measurement of NTx by ECLIA was carried out using an ECLIA automated analyzer “Picolumi III”. 20 μL each of the calibrator and sample were injected into reaction tubes. 50 μL of ruthenium-labeled S88230R antibody adjusted to a concentration of 0.1 μg/mL with the R1 reagent was injected into each reaction tube, and the resulting was stirred. 25 μL of 0.05 mg/mL Nx7 peptide-immobilized magnetic particles were injected into each reaction tube, and the resulting was allowed to react for 10.5 minutes. The liquid in the reaction tube was removed by suction, and the magnetic particles were washed with 350 μL of Picolumi BF washing solution (manufactured by Sekisui Medical Co., Ltd.). 300 μL of a luminescent electrolytic solution (manufactured by Sekisui Medical Co., Ltd.) was injected into the reaction tube, and the beads were guided to a flow cell electrode to measure the luminescence. From the measurement results of the calibrators, a calibration curve was created using the Logit-Log linear equation, and the measurement values for the samples were calculated. In this process, the measured value of the sample diluted with urine was calculated by subtracting the NTx value derived from the urine used for dilution from the actual measured value.

### Analysis Example 2 Measurement Procedure Using Osteomark

NTx was measured using Osteomark kit for cross-linked N-telopeptides of type I collagen (Abbott Diagnostics Medical Co., Ltd.) according to the product's package insert. The standard product included in the kit was used, and the sample used was the same as that used for the ECLIA measurement. In this process, the measured value of the sample diluted with urine was calculated by subtracting the NTx value derived from the urine used for dilution from the actual measured value.

### Comparative Example 1 Dilution Recovery Test in the Case of Using Urine or Physiological Saline With Known NTx Concentration as Sample Diluent

Four urine specimens (BizCom Japan, Inc.) from osteoporosis patients were diluted 10 times with urine or physiological saline with known NTx concentration, and the dilution recovery rates when measuring the NTx concentration by ECLIA (measured value×dilution ratio/measured value for undiluted solution×100%) were determined and are shown in Table 1. With respect to some combinations of urine specimens and diluents, and physiological saline which has traditionally been widely used as a sample diluent, it has been found that the NTx measurement value fluctuates by more than 40% during dilution measurement, and measurement accuracy cannot be secured. It has been suggested that in order to obtain accurate measurement values when urine is used as a diluent for sample, it is necessary to appropriately select the urine for dilution.

TABLE 1

| | | Urine | Urine | Urine | Urine |
|---|---|---|---|---|---|
| | | sam- | sam- | sam- | sam- |
| | Sample | ple 1 | ple 2 | ple 3 | ple 4 |
| | NTx measurement | 1169 | 1175 | 32.2 | 57.6 |
| | for undiluted | | | | |
| | solution [ng/ml] | | | | |
| Diluent | Urine for dilution A | 106.9% | 102.9% | NT | NT |
| solution | Urine for dilution B | NT | NT | 143.5% | 103.3% |
| | Urine for dilution C | NT | NT | 156.5% | 171.1% |
| | Physiological saline | 209.5% | 156.8% | NT | NT |

### Example 1 Dilution Recovery Test Using Sample Diluents Containing Various Additives

Various diluents were prepared by adding various additives to 20 mM HEPES with a pH of 6.5. Table 2 shows the dilution recovery rates when two urine specimens were diluted 10 times with the prepared diluents and the NTx concentrations was measured by ECLIA. When using a diluent containing uric acid at a concentration of 0.08 to 0.25%, the dilution recovery rates were good, within 75 to 125%. On the other hand, the dilution recovery rates were poor when no additive was added or when urea and creatinine, which are also contained in urine like uric acid, were added to the diluents. From the above, it has become clear that NTx dilution measurement can be carried out accurately by adding uric acid to the diluent.

TABLE 2

| | Urine | Urine |
|---|---|---|
| Sample | sample 5 | sample 6 |

| NTx measurement for undiluted | | | 380 | 430 |
|---|---|---|---|---|
| solution [ng/ml] | | | | |
| | Additive | None | 151.2% | 147.0% |
| | for | 0.08% Uric acid | 121.9% | 122.4% |
| | diluent | 0.15% Uric acid | 103.0% | 107.8% |
| | | 0.20% Uric acid | 86.0% | 81.2% |
| | | 0.25% Uric acid | 76.2% | 77.9% |
| | | 2% Urea | 147.6% | 139.7% |
| | | 0.2% creatinine | 157.3% | 147.1% |

### Example 2 Correlation Between Dilution Recovery Test Using Sample Diluent Containing Various Additives and Measurement Using Osteomark

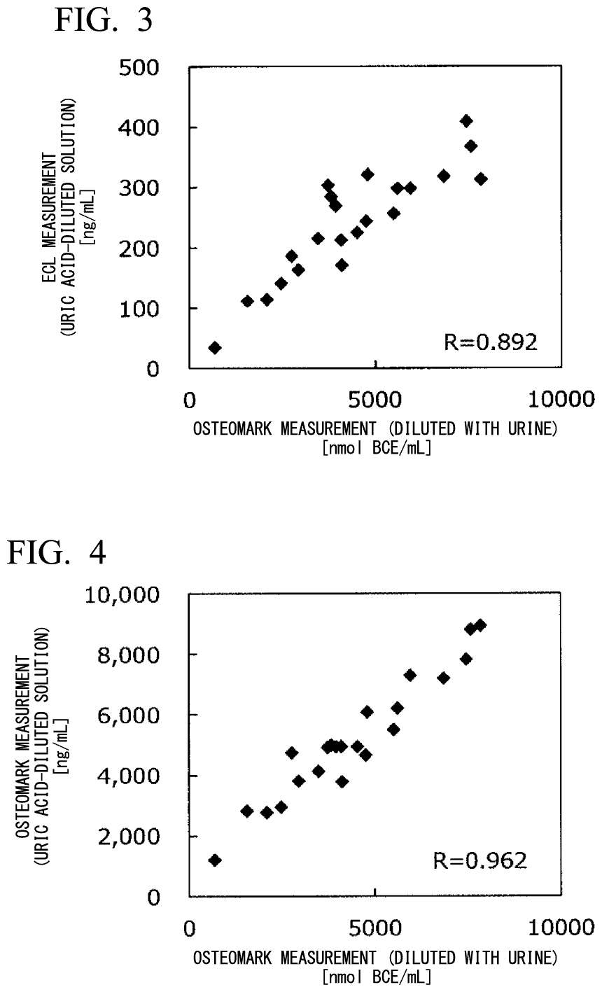

22 urine specimens from osteoporosis patients were diluted 10 times with urine with a good dilution recovery rate or a diluent containing uric acid (0.15% uric acid, 20 mM HEPES, 150 mM NaCl, pH 7.6), and subjected to ECLIA measurement and Osteomark measurement. FIG. 2 shows the correlation between the ECLIA measurement values and the Osteomark measurement values when dilution measurement was performed using urine. FIG. 3 shows the correlation between the ECLIA measurement values and the Osteomark measurement values when dilution measurement was performed using a diluent containing uric acid of this Example. The correlation coefficients between the Osteomark measurement values and the ECLIA measurement values were 0.909 for the dilution measurement using urine, and 0.892 for the dilution measurement using a diluent containing uric acid. Therefore, the measurement results were equivalent between the diluent solutions, and it was found that dilution measurement using a diluent containing uric acid is possible regardless of the sample type. For the Osteomark measurement, the results of comparing the measured values between dilution with urine and dilution with a diluent containing uric acid are shown in FIG. 4. The correlation coefficient between the diluent solutions was 0.962, indicating that the diluent solution containing uric acid of this Example can also be used in measurement methods other than ECLIA measurement. Using a diluent containing uric acid as the diluent solution makes it possible to omit division by the NTx value derived from the urine for dilution as well as selection of appropriate urine for dilution, which were conventionally necessary when urine was used as a sample diluent. This allows the dilution measurement of the NTx value to be carried out more easily.

### INDUSTRIAL APPLICABILITY

The present invention can provide a reagent for measuring NTx, which is easier to handle and enables NTx measurement with higher accuracy.

1. A measurement reagent for a cross-linked N-telopeptide of type I collagen containing uric acid or its salt.

2. The measurement reagent according to claim 1, which is a reagent for immunoassay and is in a liquid form.

3. The measurement reagent according to claim 1, which is a measurement reagent for measuring a cross-linked N-telopeptide of type I collagen in urine.

4. The measurement reagent according to claim 1, which is a sample diluent, an antibody diluent, or a particle suspension.

5. The measurement reagent according to claim 1, wherein a concentration of uric acid or its salt is 0.001 to 10% by mass, based on the measurement reagent.

6. The measurement reagent according to claim 1, which further comprises a buffer.

7. An immunoassay method for a cross-linked N-telopeptide of type I collagen, comprising:
a contacting step of bringing a biological sample into contact with uric acid or its salt, or a reagent containing uric acid or its salt.

8. The immunoassay method according to claim 7, wherein the contacting step is a step of bringing the biological sample into contact with the reagent containing uric acid or its salt.

9. The immunoassay method according to claim 7, wherein the biological sample is urine.

10. The immunoassay method according to claim 7, which further comprises:
a step of bringing the biological sample into contact with an antibody that binds to a cross-linked N-telopeptide of type I collagen or an antibody fragment thereof; and
a step of measuring a signal derived from a labeling substance indirectly or directly bound to the antibody or the antibody fragment thereof.

11. The immunoassay method according to claim 7, wherein a concentration of uric acid during antibody-antigen reaction is 0.0001 to 1% by mass.

12. The immunoassay method according to claim 7, which does not comprise a step of dividing the signal or a measured value of the cross-linked N-telopeptide of type I collagen based on the signal by an amount of another cross-linked N-telopeptide of type I collagen derived from a source other than the biological sample, or subtracting the amount of the another cross-linked N-telopeptide of type I collagen from the signal or the measured value of the cross-linked N-telopeptide of type I collagen.

13. A method for preparing a measurement reagent for a cross-linked N-telopeptide of type I collagen, comprising:
adding uric acid or its salt to a solvent.

14. The method according to claim 13, wherein the solvent is a buffer.


SEQUENCE LISTING (2 sequences)

SEQ ID NO: 1 - protein, 8 aa, synthetic construct
  REGION 1..8: note = Peptide
  MOD_RES 1: note = pyroglutamic acid
XYDGKGVG

SEQ ID NO: 2 - protein, 9 aa, synthetic construct
  REGION 1..9: note = PEPTIDE
  PEPTIDE 6..7: note = The sixth Cys and the seventh Gly are both bound to the fifth Lys.
QYDGKCGVG